SEQUENCE LISTING (20 sequences)

SEQ ID NO: 1 - protein, 118 aa, synthetic construct
  REGION 1..118: note = BAN0805 VH
QVQLQESGPGLVKPSETLSLTCTVSGFSLTSYGVHWIRQPPGKGLEWIGVIWRGGSTDYSAAFMSRLTISKDTSKNQVSLKLSSVTAADTAVYYCAKLLRSVGGFADWGQGTMVTVSS

SEQ ID NO: 2 - protein, 112 aa, synthetic construct
  REGION 1..112: note = BAN0805 VL
DIVMTQSPLSLPVTPGEPASISCRSSQTIVHNNGNTYLEWYLQKPGQSPQLLIYKVSNRFSGVPDRFSGSGSGTDFTLKISRVEAEDVGVYYCFQGSHVPFTFGQGTKLEIK

SEQ ID NO: 3 - protein, 444 aa, synthetic construct
  REGION 1..444: note = BAN0805 Heavy Chain
QVQLQESGPGLVKPSETLSLTCTVSGFSLTSYGVHWIRQPPGKGLEWIGVIWRGGSTDYSAAFMSRLTISKDTSKNQVSLKLSSVTAADTAVYYCAKLLRSVGGFADWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 4 - protein, 219 aa, synthetic construct
  REGION 1..219: note = BAN0805 Light Chain
DIVMTQSPLSLPVTPGEPASISCRSSQTIVHNNGNTYLEWYLQKPGQSPQLLIYKVSNRFSGVPDRFSGSGSGTDFTLKISRVEAEDVGVYYCFQGSHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 10 aa, synthetic construct
  REGION 1..10: note = BAN0805 VH-CDR-1 (Chothia)
GFSLTSYGVH

SEQ ID NO: 6 - protein, 14 aa, synthetic construct
  REGION 1..14: note = BAN0805 VH-CDR-2 (Chothia)
VIWRGGSTDYSAAF

SEQ ID NO: 7 - protein, 10 aa, synthetic construct
  REGION 1..10: note = BAN0805 VH-CDR-3 (Kabat/Chothia)
LLRSVGGFAD

SEQ ID NO: 8 - protein, 16 aa, synthetic construct
  REGION 1..16: note = BAN0805 VL-CDR-1 (Kabat/Chothia)
RSSQTIVHNNGNTYLE

SEQ ID NO: 9 - protein, 7 aa, synthetic construct
  REGION 1..7: note = BAN0805 VL-CDR-2 (Kabat/Chothia)
KVSNRFS

SEQ ID NO: 10 - protein, 9 aa, synthetic construct
  REGION 1..9: note = BAN0805 VL-CDR-3 (Kabat/Chothia)
FQGSHVPFT

SEQ ID NO: 11 - DNA, 1395 bp, synthetic construct
  misc_feature 1..1395: note = BAN0805 HC gene with signal sequence
  source 1..1395: mol_type = other DNA
atggaatggtcctgggtgttcctgttcttcctgtccgtgaccaccggcgtgcactctcaggttcagctgcaagagtctggccctggcctggtcaagccttccgaaacactgtctctgacctgcaccgtgtccggcttctccctgacatcttatggggtgcactggatcagacagcctccaggcaaaggcctggaatggatcggagtgatttggagaggcggctccaccgattactccgccgccttcatgtcccggctgaccatctctaaggacacctccaagaaccaggtgtccctgaagctgtcctctgtgaccgctgctgataccgccgtgtactactgtgccaagctgctgagatctgtcggcggctttgctgattggggccagggcacaatggtcaccgtgtctagcgcttctacaaagggcccaagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggctgatga

SEQ ID NO: 12 - DNA, 723 bp, synthetic construct
  misc_feature 1..723: note = BAN0805 LC gene with signal sequence
  source 1..723: mol_type = other DNA
atgtctgtgcctacacaggttctgggactgctgctgctgtggctgaccgacgccagatgcgacatcgtgatgacccagtctccactgagcctgcctgtgacacctggcgagcctgcttccatctcctgcagatcctctcagaccatcgtgcacaacaacggcaacacctacctggaatggtatctgcagaagcccggccagtctcctcagctgctgatctacaaggtgtccaaccggttctctggcgtgcccgacagattttccggctctggctctggcaccgacttcaccctgaagatctccagagtggaagccgaggacgtgggcgtgtactactgcttccaaggctctcacgtgcccttcacctttggccagggcaccaagctggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgctgatga

SEQ ID NO: 13 - DNA, 413 bp, synthetic construct
  misc_feature 1..413: note = BAN0805 VH gene with signal sequence
  source 1..413: mol_type = other DNA
atggaatggtcctgggtgttcctgttcttcctgtccgtgaccaccggcgtgcactctcaggttcagctgcaagagtctggccctggcctggtcaagccttccgaaacactgtctctgacctgcaccgtgtccggcttctccctgacatcttatggggtgcactggatcagacagcctccaggcaaaggcctggaatggatcggagtgatttggagaggcggctccaccgattactccgccgccttcatgtcccggctgaccatctctaaggacacctccaagaaccaggtgtccctgaagctgtcctctgtgaccgctgctgataccgccgtgtactactgtgccaagctgctgagatctgtcggcggctttgctgattggggccagggcacaatggtcaccgtgtctagcgc

SEQ ID NO: 14 - DNA, 396 bp, synthetic construct
  misc_feature 1..396: note = BAN0805 VL gene with signal sequence
  source 1..396: mol_type = other DNA
atgtctgtgcctacacaggttctgggactgctgctgctgtggctgaccgacgccagatgcgacatcgtgatgacccagtctccactgagcctgcctgtgacacctggcgagcctgcttccatctcctgcagatcctctcagaccatcgtgcacaacaacggcaacacctacctggaatggtatctgcagaagcccggccagtctcctcagctgctgatctacaaggtgtccaaccggttctctggcgtgcccgacagattttccggctctggctctggcaccgacttcaccctgaagatctccagagtggaagccgaggacgtgggcgtgtactactgcttccaaggctctcacgtgcccttcacctttggccagggcaccaagctggaaatcaag

SEQ ID NO: 15 - protein, 5 aa, synthetic construct
  REGION 1..5: note = BAN0805 VH-CDR-1 (Kabat)
SYGVH

SEQ ID NO: 16 - protein, 16 aa, synthetic construct
  REGION 1..16: note = BAN0805 VH-CDR-2 (Kabat)
VIWRGGSTDYSAAFMS

SEQ ID NO: 17 - DNA, 1335 bp, synthetic construct
  misc_feature 1..1335: note = BAN0805 HC gene
  source 1..1335: mol_type = other DNA
caggttcagctgcaagagtctggccctggcctggtcaagccttccgaaacactgtctctgacctgcaccgtgtccggcttctccctgacatcttatggggtgcactggatcagacagcctccaggcaaaggcctggaatggatcggagtgatttggagaggcggctccaccgattactccgccgccttcatgtcccggctgaccatctctaaggacacctccaagaaccaggtgtccctgaagctgtcctctgtgaccgctgctgataccgccgtgtactactgtgccaagctgctgagatctgtcggcggctttgctgattggggccagggcacaatggtcaccgtgtctagcgcttctacaaagggcccaagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggctga

SEQ ID NO: 18 - DNA, 660 bp, synthetic construct
  misc_feature 1..660: note = BAN0805 LC gene
  source 1..660: mol_type = other DNA
gacatcgtgatgacccagtctccactgagcctgcctgtgacacctggcgagcctgcttccatctcctgcagatcctctcagaccatcgtgcacaacaacggcaacacctacctggaatggtatctgcagaagcccggccagtctcctcagctgctgatctacaaggtgtccaaccggttctctggcgtgcccgacagattttccggctctggctctggcaccgacttcaccctgaagatctccagagtggaagccgaggacgtgggcgtgtactactgcttccaaggctctcacgtgcccttcacctttggccagggcaccaagctggaaatcaagcgtacggtggccgctcccagcgtgttcatcttccccccaagcgacgagcagctgaagagcggcaccgccagcgtggtgtgtctgctgaacaacttctaccccagggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaacaggggcgagtgctga

SEQ ID NO: 19 - DNA, 356 bp, synthetic construct
  misc_feature 1..356: note = BAN0805 VH gene
  source 1..356: mol_type = other DNA
caggttcagctgcaagagtctggccctggcctggtcaagccttccgaaacactgtctctgacctgcaccgtgtccggcttctccctgacatcttatggggtgcactggatcagacagcctccaggcaaaggcctggaatggatcggagtgatttggagaggcggctccaccgattactccgccgccttcatgtcccggctgaccatctctaaggacacctccaagaaccaggtgtccctgaagctgtcctctgtgaccgctgctgataccgccgtgtactactgtgccaagctgctgagatctgtcggcggctttgctgattggggccagggcacaatggtcaccgtgtctagcgc

SEQ ID NO: 20 - DNA, 336 bp, synthetic construct
  misc_feature 1..336: note = BAN0805 VL gene
  source 1..336: mol_type = other DNA
gacatcgtgatgacccagtctccactgagcctgcctgtgacacctggcgagcctgcttccatctcctgcagatcctctcagaccatcgtgcacaacaacggcaacacctacctggaatggtatctgcagaagcccggccagtctcctcagctgctgatctacaaggtgtccaaccggttctctggcgtgcccgacagattttccggctctggctctggcaccgacttcaccctgaagatctccagagtggaagccgaggacgtgggcgtgtactactgcttccaaggctctcacgtgcccttcacctttggccagggcaccaagctggaaatcaag

(19) United States
(12) Patent Application Publication
(10) Pub. No.: US 2026/0217805 A1
(43) Pub. Date: Jul. 30, 2026

(54) ALPHA-SYNUCLEIN PROTOFIBRIL-BINDING ANTIBODIES

(71) Applicant: BioArctic AB, Stockholm (SE)

(72) Inventors: Eva Nordström, Upplands Väsby (SE); Jessica Sigvardson, Spånga (SE); Patrik Nygren, Stockholm (SE)

(21) Appl. No.: 19/355,556

(22) Filed: Oct. 10, 2025

Related U.S. Application Data
(63) Continuation of application No. 17/726,144, filed on Apr. 21, 2022, now abandoned.
Application No. 17/726,144 is a continuation of application No. 17/359,212, filed on Jun. 25, 2021, now Pat. No. 11,339,212.
(60) Provisional application No. 63/044,881, filed on Jun. 26, 2020.
(60) Provisional application No. 63/071,150, filed on Aug. 27, 2020.

(51) Int. Cl. C07K 16/18

(52) U.S. Cl. CPC C07K 16/18; C07K 2317/92

(57) ABSTRACT

The present disclosure is based, in part, on the discovery of antibodies that selectively targets human α-synuclein aggregates such as oligomers/protofibrils, such as BAN0805. BAN0805 has a lower tendency to bind to the undesired monomeric α-synuclein target as compared to mouse monoclonal antibody mAb47.

### CROSS-REFERENCE TO RELATED APPLICATIONS

This application is a continuation of U.S. patent application Ser. No. 17/726,144, filed Apr. 21, 2022, which is a continuation of U.S. patent application Ser. No. 17/359,212, filed Jun. 25, 2021, now granted as U.S. Pat. No. 11,339,212, which claims benefit to U.S. Provisional Application No. 63/044,881, filed Jun. 26, 2020, and U.S. Provisional Application No. 63/071,150, filed Aug. 27, 2020, the content of each of which is incorporated by reference herein in its entirety.

### REFERENCE TO SEQUENCE LISTING

The instant application contains a Sequence Listing which has been submitted electronically in XML file format and is hereby incorporated by reference in its entirety. Said XML copy, created on Mar. 30, 2026, is named BAR-71903_SL.xml and is 32,498 bytes in size.

### BACKGROUND

International Patent Application No. WO2011/104696 A1 (which is incorporated herein by reference) discloses a murine monoclonal IgG antibody mAb47, which binds to protofibril forms of α-synuclein. There remains a need for antibodies that selectively bind to protofibril forms of α-synuclein that are suitable for use in humans.

### SUMMARY

The present disclosure relates to antibodies having high affinity for human α-synuclein protofibrils and low affinity for α-synuclein monomers. In some embodiments, the antibodies described herein selectively target human α-synuclein aggregates such as oligomers/protofibrils, i.e., with a much stronger binding to α-synuclein protofibrils compared to monomer. In some embodiments, the antibodies described herein have better selectivity than mAb47 when comparing the α-synuclein protofibril versus monomer binding ratios. In some embodiments, the antibodies described herein are anti-α-synuclein antibodies.

In one aspect, the present disclosure relates to BAN0805, a monoclonal antibody comprising a heavy chain comprising an amino acid sequence set forth in SEQ ID NO:3 and a light chain comprising an amino acid sequence set forth in SEQ ID NO:4 that selectively targets human α-synuclein aggregates such as oligomers/protofibrils with high affinity for human α-synuclein protofibrils and low affinity for α-synuclein monomers. Interestingly, BAN0805 also exhibits lower α-synuclein monomer binding than mAb47, resulting in better selectivity for BAN0805 than for mAb47 when comparing the α-synuclein protofibril versus monomer binding ratios. Additionally, binding to β- and γ-synuclein monomer or Aβ-protofibrils was not detected for BAN0805.

The present disclosure further relates to antibodies for improvements in treating neurodegenerative disorders with α-synuclein pathology, including, but not limited to, Parkinson's disease (PD).

### BRIEF DESCRIPTION OF THE DRAWINGS

FIG. 1 shows heat stress data for BAN0805. Samples of the purified candidate antibodies at 1 mg/mL were exposed to temperatures of a) 4° C., b) 25° C., c) 37° C. and d) 50° C. for two weeks. Samples were then analyzed by SEC-MALS to check for aggregation. The data suggest there are no aggregation concerns for BAN0805 due to heat stress.

FIG. 2 shows the inhibition ELISA with IC₅₀ values for BAN0805 when bound to α-synuclein monomers and protofibrils (PF). BAN0805 has a 910-fold better selectivity for the protofibril form of α-synuclein compared to mAb47 which only has a 340-fold selectivity (not shown). The protofibril level was expressed as equivalent to monomer level in concentration and the size of the protofibrils was not considered. The fold selectivity was calculated by dividing the IC₅₀ value for the monomer binding with the IC₅₀ value for the PF binding.

FIG. 3 shows binding and selectivity for BAN0805 compared to mAb47 using Biacore SPR. The K_{D} values for α-synuclein protofibril were similar for mAb47 and BAN0805, showing that the modification of mAb47 did not affect the strong binding to α-synuclein protofibril, confirming the results from the inhibition ELISA. The K_{D} values measured with SPR resulted in a 110,000-fold and 18,000-fold selectivity for PF vs monomer for BAN0805 and mAb47, respectively. Representative sensorgrams of mAb47 and BAN0805 SPR measurements on Biacore 8K are shown.

FIG. 4 shows cross-reactivity of BAN0805, here referred to as hu47-IgG4 to α-synuclein monomer, β-synuclein monomer, γ-synuclein monomer and Aβ-protofibril using inhibition ELISA. The result showed no detectable binding to β- or γ-synuclein monomer or Aβ-protofibril.

### DETAILED DESCRIPTION

The present disclosure relates to antibodies having high affinity for human α-synuclein protofibrils and low affinity for α-synuclein monomers.

As disclosed herein, the present disclosure relates to the following embodiments.

Embodiment 1. An antibody comprising a heavy chain comprising the amino acid sequence of SEQ ID NO: 1 and a light chain comprising the amino acid sequence of SEQ ID NO: 2.

Embodiment 2. The antibody of embodiment 1, wherein the antibody is of the IgG isotype.

Embodiment 3. The antibody of embodiment 1, wherein the antibody is of the IgG4 isotype.

Embodiment 4. The antibody of any one of embodiments 1-3, wherein the antibody has a K_{D} value for binding the protofibril form of α-synuclein at least 110,000 times smaller than the K_{D} value for binding the monomeric form of α-synuclein.

Embodiment 5. The antibody of any one of embodiments 1-3, wherein the antibody has a K_{D} value for binding the protofibril form of α-synuclein of at most 18 μM and a K_{D} value for binding the monomeric form of α-synuclein of at least 2200 nM.

Embodiment 6. The antibody of either embodiment 4 or 5, wherein the K_{D} of said antibody for binding to the protofibril form of α-synuclein and the K_{D} of said antibody for binding to the monomeric form of α-synuclein are measured by SPR.

Embodiment 7. An antibody comprising a heavy chain comprising the amino acid sequence of SEQ ID NO: 3 and a light chain comprising the amino acid sequence of SEQ ID NO: 4.

Embodiment 8. The antibody of embodiment 7, wherein the antibody comprises two heavy chains and two light chains.

Embodiment 9. A nucleic acid encoding a polypeptide comprising an amino acid sequence selected from the group consisting of SEQ ID NOs: 1-4.

Embodiment 10. The nucleic acid of embodiment 9, comprising a sequence selected from the group consisting of SEQ ID NOs: 11-14 and 17-20.

Embodiment 11. One or more nucleic acids encoding the antibody of any one of embodiments 1 to 8.

Embodiment 12. The one or more nucleic acids of embodiment 11, wherein
(a) the one or more nucleic acids comprise the sequences of SEQ ID NOs: 11 and 12,
(b) the one or more nucleic acids comprise the sequences of SEQ ID NOs: 13 and 14,
(c) the one or more nucleic acids comprise the sequences of SEQ ID NOs: 17 and 18, or
(d) the one or more nucleic acids comprise the sequences of SEQ ID NOs: 19 and 20.

Embodiment 13. One or more vector(s) comprising the nucleic acid(s) of any one of embodiments 9, 10, 11 or 12.

Embodiment 14. A host cell comprising the nucleic acid(s) of any one of embodiments 9 to 12.

Embodiment 15. A host cell comprising the one or more vector(s) of embodiment 13.

Embodiment 16. A host cell expressing the antibody of any one of embodiments 1-8.

Embodiment 17. A composition comprising at least one antibody of any one of embodiments 1-8, and a pharmaceutically acceptable carrier.

In one aspect, the present disclosure relates to an antibody having a high affinity for human α-synuclein protofibrils and low affinity of α-synuclein monomers, and comprising a heavy chain comprising the amino acid sequence of SEQ ID NO: 1, and a light chain comprising the amino acid sequence of SEQ ID NO: 2.

In one embodiment, the antibodies provided herein comprise a heavy chain comprising the amino acid sequence of SEQ ID NO: 1 and a light chain comprising the amino acid sequence of SEQ ID NO: 2.

In one embodiment, the antibodies provided herein comprise a heavy chain comprising the amino acid sequence of SEQ ID NO:3 and a light chain comprising the amino acid sequence of SEQ ID NO:4. In some embodiments, the antibodies provided herein comprise two heavy chains and two light chains.

In one embodiment, the antibody described in the present disclosure is of the IgG isotype, in particular human IgG isotype. In another embodiment, the antibody is of the IgG4 isotype.

Within the present disclosure, high affinity to human α-synuclein protofibrils refers to a dissociation constant K_{D} of less than 10⁻⁷ M for human α-synuclein protofibrils. Accordingly, in one embodiment, the antibodies described in the present disclosure have a K_{D} of less than 10⁻⁸, 10⁻⁹, 10⁻¹⁰, 10⁻¹¹ M, or 10⁻¹² M for human α-synuclein protofibrils. In specific embodiments, the antibodies comprise a heavy chain comprising an amino acid sequence set forth in SEQ ID NO: 1 and a light chain comprising an amino acid sequence set forth in SEQ ID NO: 2, and have a K_{D} of 11.2 to 25.8 μM for human α-synuclein protofibrils.

In another embodiment, the antibodies comprise a heavy chain comprising the amino acid sequence of SEQ ID NO: 1 and a light chain comprising the amino acid sequence of SEQ ID NO: 2, and have low affinity to human α-synuclein monomer. For example, the K_{D} of the antibodies described in the present disclosure for binding to the monomeric form of α-synuclein is at least 1500 nM, at least 1600 nM, at least 1700 nM, at least 1800 nM, at least 1900 nM, at least 2000 nM, at least 2100 nM, at least 2200 nM, at least 2300 nM, at least 2400 nM, at least 2500 nM, at least 2600 nM, at least 2700 nM, at least 2800 nM, at least 2900 nM, or at least 3000 nM. In specific embodiments, the antibodies comprise a heavy chain comprising an amino acid sequence set forth in SEQ ID NO: 1 and a light chain comprising an amino acid sequence set forth in SEQ ID NO: 2, and have a K_{D} of 1650 nM to 2730 nM for the human α-synuclein monomer.

In one embodiment, the antibodies comprise a heavy chain comprising the amino acid sequence of SEQ ID NO: 1 and a light chain comprising the amino acid sequence of SEQ ID NO: 2, and have greater than 80,000 fold, greater than 90,000 fold, greater than 100,000 fold, greater than 110,000 fold, or greater than 120,000 fold selectivity to human α-synuclein protofibril versus monomeric α-synuclein. In specific embodiments, the antibodies comprise a heavy chain comprising an amino acid sequence set forth in SEQ ID NO: 1 and a light chain comprising an amino acid sequence set forth in SEQ ID NO: 2, and have a 64,000 fold to 244,000 fold selectivity to human α-synuclein protofibril versus monomeric α-synuclein.

In one embodiment, these binding affinities are measured using inhibition ELISA, for example, as described in example 3. In another embodiment, these binding affinities are measured by Surface Plasmon Resonance (SPR), for example, as described in example 3.

In another aspect, provided herein are nucleic acids encoding at least one polypeptide comprising an amino acid sequence selected from the group consisting of SEQ ID NOs: 1-4. The nucleic acid can be DNA or RNA. The nucleic acid may comprise a sequence selected from the group consisting of SEQ ID NOs: 11-14 and 17-20.

In another aspect, provided herein are one or more nucleic acids encoding an antibody of the invention. In one embodiment, the one or more nucleic acids comprise the sequences of SEQ ID Nos: 11 and 12. In another embodiment, the one or more nucleic acids comprise the sequences of SEQ ID Nos: 13 and 14. In one embodiment, the one or more nucleic acids comprise the sequences of SEQ ID Nos: 17 and 18. In one embodiment, the one or more nucleic acids comprise the sequences of SEQ ID Nos: 19 and 20.

In another aspect, provided herein are vectors comprising nucleic acids that encode at least one polypeptide comprising an amino acid sequence selected from the group consisting of SEQ ID NOs: 1-4. Such vectors include but are not limited to cloning vectors or expression vectors. In one aspect, one or more vectors are providing encoding an antibody of the invention. In one embodiment, the one or more vectors comprise the sequences of SEQ ID Nos: 11 and 12. In another embodiment, the one or more vectors comprise the sequences of SEQ ID Nos: 13 and 14. In one embodiment, the one or more vectors comprise the sequences of SEQ ID Nos: 17 and 18. In one embodiment, the one or more vectors comprise the sequences of SEQ ID Nos: 19 and 20.

In another aspect, provided herein are host cells comprising nucleic acids that encode at least one polypeptide comprising an amino acid sequence selected from the group consisting of SEQ ID NOs: 1-4. In one embodiment, the host cells comprise nucleic acid(s) encoding an antibody of the invention. The host cells described herein may be mammalian cells, such as B cells, hybridomas, or CHO cells. In one embodiment, the host cells described herein are human cells.

In another aspect, provided herein is a composition comprising an antibody of the invention and a pharmaceutically acceptable excipient.

### EXAMPLES

### Example 1—Generation of Antibody Candidates

The initial variants of mAb47 were generated by direct grafting of mAb47 CDRs to human framework sequences, and making back-mutations to the mouse residues at various positions. None of the initial variants showed desired binding properties to α-synuclein. Therefore, a new model was built and analyzed to find more possible mutations for generating new variants.

In the second attempt to improve mAb47, less likely residues that interact with the target and the residues at 4 Å from the CDRs determined were checked. An antibody variant having a heavy chain sequence set forth in SEQ ID NO: 3 and a light chain sequence set forth in SEQ ID NO: 4 was generated and named as BAN0805. It was found that the back mutations V71K and R94K, while simultaneously present in BAN0805, are crucial for the binding capacity of these antibodies as their removal in the other variants have resulted in a loss of binding.

Since BAN0805 has one less back mutation than a comparable variant, and has shown binding and selectivity for protofibrils over monomer species, BAN0805 was chosen as the lead candidate.

### Example 2—Characterization of Antibody Candidates

To determine thermal stability, the antibodies were subjected to higher temperatures for 10 minutes, cooled to 4° C. and used in an ELISA assay at the EC₈₀ concentration of each candidate (usually 5-50 ng/mL). BAN0805 was stable, retaining its binding ability to α-synuclein up to 75° C. where it started to decrease, while the chimeric mouse antibody c47 or cmAb47 (the chimera combining human IgG4 and the variable region of mAb47) binding dropped drastically around 5° C. earlier.

In order to determine the melting temperature of the antibodies, cmAb47 was tested against BAN0805 in a thermal shift assay. Melting temperature data indicates that the Tm for BAN0805 was calculated to be 65-65.4° C., lower than the chimeric at 70° C.

Additionally, purified samples at 1 mg/mL were injected at 0.4 mL/min into a size exclusion column in an HPLC system and analyzed by multi-angle light scattering to determine the absolute molar masses and check for aggregation. The data suggest there were no aggregation concerns for BAN0805. BAN0805 was monodispersed (Mw/Mn<1.05). The mass recovery was 100% (calculated mass over injected mass), which indicates good protein recovery.

Cross-Interaction Chromatography using bulk purified human polyclonal IgG is a technique for monitoring non-specific protein-protein interactions, and can be used to discriminate between soluble and insoluble antibodies. An elevated Retention Index (k′) indicates a self-interaction propensity and a low solubility. BAN0805 showed a Retention Index of 0.025 which is below 0.035 of the cmAb47, indicating a low propensity for non-specific interactions and good solubility.

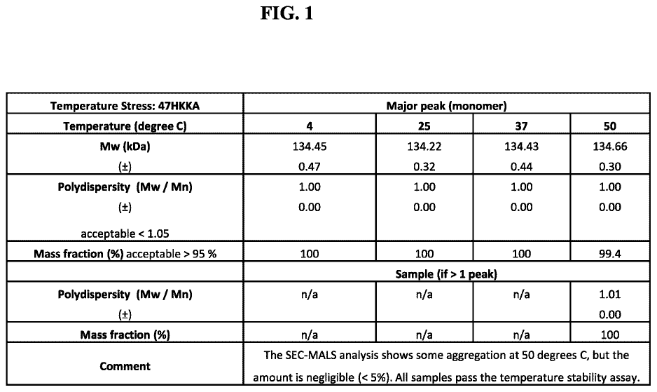

For freeze/thaw stress analysis, samples of the purified candidate antibodies at 1 mg/mL were subjected to 10 cycles of 15 minutes at −80° C. followed by thawing for 15 minutes at Room Temperature. For heat-induced stress analysis, samples of the purified candidate antibodies at 1 mg/mL were exposed to temperatures of a) 4° C., b) 25° C., c) 37° C. and d) 50° C. for two weeks. Samples were then analyzed by SEC-MALS to check for aggregation. The data suggest that freeze thaw cycles and heat stress did not cause aggregation in BAN0805. See FIG. 1.

BAN0805 was analyzed and compared to the closest germline (IGVH4-59*03/IGHJ3*01 for HK and IGVK2-28*01/IGKJ2*02 for KA) following IMGT CDR definitions and the DomainGapAlign tool. Overall identity to human germline was 86.5% for the light chain, above the 85% cutoff for it to be considered humanized for this analysis. For the heavy chain, after grafting CDRs and introducing two mouse back mutations, the percentage identity to human germline dropped to just under 81%. This might be explained by the fact that the IMGT CDR2 is significantly shorter than the Kabat definition used here, which caused the insertion of a higher number of mouse residues at the beginning of the framework 3.

### Example 3—Selective Binding of BAN0805 to Human α-Synuclein Protofibrils

Binding selectivity of BAN0805 to human α-synuclein protofibrils were measured by both inhibition ELISA and Surface Plasmon Resonance (SPR).

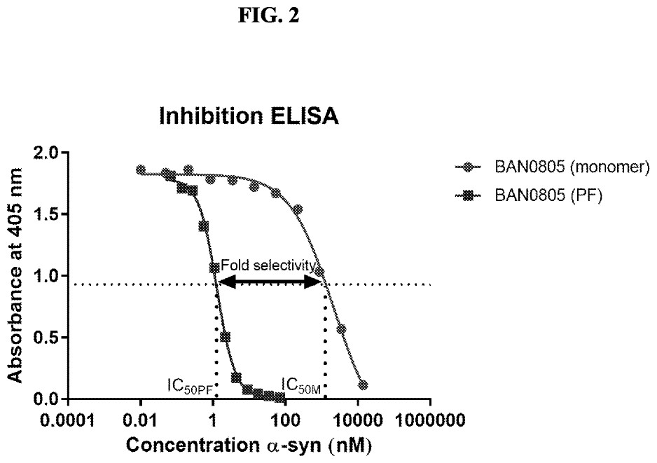

The IC₅₀ values for α-synuclein protofibril were very similar for mAb47 and BAN0805 (2.7 nM and 2.2 nM respectively) showing that the binding characteristics to protofibril did not change after humanization. In contrast, binding to α-synuclein monomer did change, resulting in a reduced binding strength of BAN0805 to α-synuclein monomer. This resulted in better selectivity for α-synuclein protofibril versus monomer for BAN0805 (910-fold) compared to mAb47 (340-fold). See FIG. 2.

However, due to detection limitations it was not possible to lower the antibody concentration further to make it possible to detect even lower IC₅₀ values and hence approach the “true” IC₅₀ value. Therefore, the IC₅₀ values presented have been obtained according to the current procedure for the inhibition ELISA which has been used for all mAb47 and BAN0805 batches, with the notion that IC₅₀ values for the protofibril are likely overestimated (i.e., the binding strength is likely underestimated). A more accurate binding and hence selectivity were obtained using SPR which is described below.

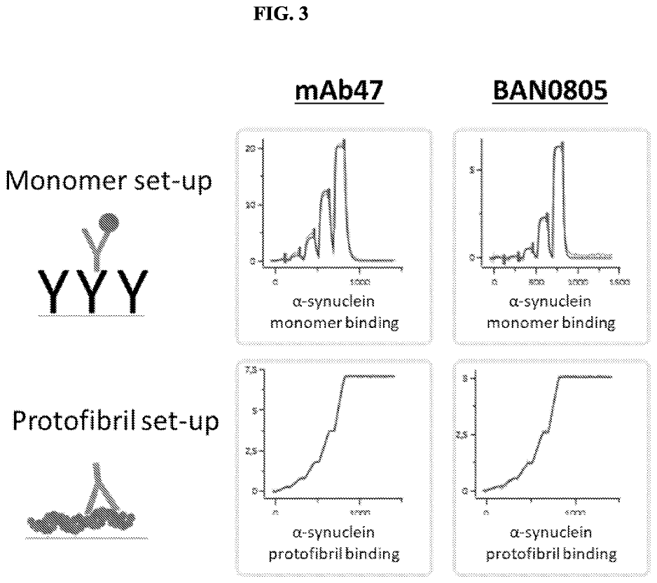

The binding selectivity of mAb47 and BAN0805 was confirmed by SPR using a Biacore 8K instrument (GE Healthcare). Due to feasibility issues caused by the complexity of the target antigen in combination with pronounced avidity dependence of the antibodies, different assay set-ups were used to assess α-synuclein protofibril and monomer binding, respectively. For measurements of binding to monomer, the chip was coated with an anti-mouse or anti-human antibody for mAb47 and BAN0805, respectively. 0.25-1.5 μg/ml mAb47 or BAN0805 was then captured on the surface, followed by a 5-fold dilution single cycle kinetics injection of α-synuclein monomer. To measure binding to protofibril (PF), the chip was coated with 0.5 μg/ml PF and a 2-fold dilution of mAb47 or BAN0805 was injected using single cycle kinetics. Representative sensorgrams for mAb47 and BAN0805 are shown in FIG. 3.

The K_{D} values for α-synuclein protofibril were similar for mAb47 and BAN0805, showing that the modification of mAb47 did not affect the strong binding to α-synuclein protofibril (Table 1), confirming the results from the inhibition ELISA. However, the K_{D} values were in the pM range, confirming the afore-mentioned limitations with the inhibition ELISA. Importantly, it was confirmed by the SPR that the affinity of BAN0805 for α-synuclein monomer was reduced in comparison to mAb47. The K_{D} values measured with SPR resulted in a 110,000-fold and 18,000-fold selectivity for PF versus monomer for BAN0805 and mAb47, respectively. Average K_{D} values for mAb47 and BAN0805 for α-synuclein monomer and protofibril are shown in Table 1.

TABLE 1
K_{D} values for mAb47 and BAN0805 for binding to α-synuclein
protofibril (PF) and monomer (M) by Biacore SPR.

| | | | | | Selectivity PF |
|---|---|---|---|---|---|
| | α-synuclein | | α-synuclein | | vs. monomeric |
| | PF K_{D} | | monomer K_{D} | | α-synuclein |
| Antibody | (pM) | | (nM) | | (fold) |
| BAN0805 | 18.5 ± 7.3 | (n = 27) | 2190 ± 540 | (n = 59) | 110 000 |
| mAb47 | 16.8 ± 8.0 | (n = 18) | 307 ± 35 | (n = 23) | 18 000 |

Data are presented as Mean ± Standard deviation (n = Number of experiments)
K_{D}: Dissociation constant

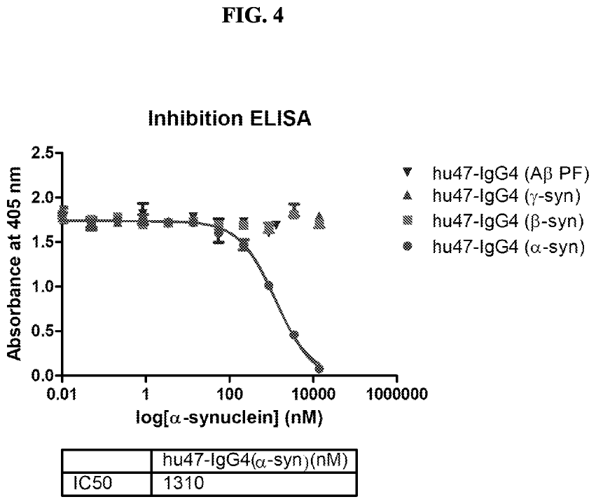

The cross-reactivity of homologous proteins, such as β- or γ-synuclein, and other aggregation prone proteins like Aβ was tested using both direct ELISA (where dense coating mimics aggregated forms of the coated protein), as well as inhibition ELISA. Here, the cross-reactivity of mAb47 and BAN0805, was analyzed side-by-side by inhibition ELISA. The inhibition ELISA was performed with β-synuclein monomer, γ-synuclein monomer and Aβ-protofibril as antigens. The result indicates there was no detectable binding of BAN0805 to β- or γ-synuclein monomer or Aβ-protofibril. A representative BAN0805 cross-reactivity test to β- or γ-synuclein in inhibition ELISA is shown in FIG. 4. Data are presented in Table 2.

TABLE 2
Cross-reactivity of mAb47 and BAN0805 to β-synuclein monomer,
γ-synuclein monomer and Aβ-protofibril.

| | β-synuclein | γ-synuclein | |
|---|---|---|---|
| | monomer | monomer | Aβ-protofibril |
| Antibody | (>14 μM) | (>14 μM) | (>5 μM) |
| BAN0805 | n.b. | n.b. | n.b. |
| mAb47 | n.b. | n.b. | n.b. |

n.b. = no binding

Results from inhibition ELISA and the Surface Plasmon Resonance (SPR) Biacore data both showed the affinity of BAN0805 for α-synuclein monomer was reduced in comparison to mAb47, indicating a better selectivity of BAN0805 compared to mAb47. Additionally, no binding to β- and γ-synuclein monomer or Aβ-protofibrils was seen at concentrations tested (up to μM range) for BAN0805.

Thus, the present disclosure relates to an antibody having high affinity for α-synuclein protofibrils and low affinity to α-synuclein monomers, and having the following characteristics compared to murine mAb47:
(1) BAN0805 has a much stronger binding to α-synuclein protofibrils compared to monomer;
(2) both inhibition ELISA and SPR Biacore data showed that the α-synuclein monomer binding was stronger for mAb47 compared to BAN0805 resulting in better selectivity for BAN0805 than for mAb47 when comparing the α-synuclein protofibril versus monomer binding ratios (i.e., BAN0805 has a lower tendency to bind to the undesired monomeric α-synuclein target as compared to mAb47); and
(3) no binding to β- and γ-synuclein monomer or Aβ-protofibrils was seen at concentrations tested (up to μM range) for BAN0805.

### Example 4—Production of BAN0805

To produce BAN0805, optimized DNA sequences encoding BAN0805 VH (SEQ ID NO: 13) and VL (SEQ ID NO: 14) including signal peptides were synthesized and cloned into GS vectors pXC-IgG4Pro(deltaK) and pXC-Kappa (Lonza), respectively. The resulting HC and LC SGVs were then used to generate a double gene vector (DGV) containing both the HC and LC genes. The optimized DNA sequences encoding BAN0805 heavy chain (HC) and light chain (LC) are set forth in SEQ ID NOs: 11 and 12, respectively. The optimized DNA sequences encoding BAN0805 heavy chain variable region (VH) and light chain variable region (VL) are set forth in SEQ ID NOs: 13 and 14, respectively. SEQ ID NOs: 11-14 all include a nucleotide sequence encoding a signal peptide (see Table 3B). The nucleotide sequences corresponding to amino acid sequences for BAN0805 HC, LC, VH, and VL excluding the signal peptide are set forth in SEQ ID NOs: 17, 18, 19 and 20, respectively. The CDR sequences of BAN0805 are listed in Table 3A. The amino acid sequences of heavy chain CDR (VH-CDR) 1-3 according to Chothia nomenclature are set forth in SEQ ID NOs: 5, 6, and 7, respectively. The amino acid sequences of heavy chain CDR (VH-CDR) 1-3 according to Kabat nomenclature are set forth in SEQ ID NOs: 15, 16, and 7, respectively. The amino acid sequence of heavy chain CDR (VH-CDR-3) according to Chothia and Kabat nomenclatures is the same and set forth in SEQ ID NO: 7. The amino acid sequences of light chain CDR (VL-CDR) 1-3 according to Chothia and Kabat nomenclatures are the same, and set forth in SEQ ID NOs: 8, 9, and 10, respectively.

The resultant DGV, termed pBAN0805/DGV, was then transiently transfected to CHOK1SV GS-KO cells, and cultured under conditions which resulted in the secretion of assembled antibody. The secreted antibody was then purified by Protein A affinity chromatography.

TABLE 3
SEQUENCE LISTING
A. BAN0805
VH:
QVQLQESGPGLVKPSETLSLTCTVSGFSLTSYGVHWIRQPPGKGLEWIGVIWRGGSTDY
SAAFMSRLTISKDTSKNQVSLKLSSVTAADTAVYYCAKLLRSVGGFADWGQGTMVTV
SS (SEQ ID NO: 1)
VL:
DIVMTQSPLSLPVTPGEPASISCRSSQTIVHNNGNTYLEWYLQKPGQSPQLLIYKVSNRF
SGVPDRFSGSGSGTDFTLKISRVEAEDVGVYYCFQGSHVPFTFGQGTKLEIK (SEQ ID
NO: 2)
Heavy Chain
QVQLQESGPGLVKPSETLSLTCTVSGFSLTSYGVHWIRQPPGKGLEWIGVIWRGGSTD
YSAAFMSRLTISKDTSKNQVSLKLSSVTAADTAVYYCAKLLRSVGGFADWGQGTMVT
VSSASTKGPSVFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCPPCPAPEFLGG
PSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDK
SRWQEGNVFSCSVMHEALHNHYTQKSLSLSLG (SEQ ID NO: 3)
Light Chain
DIVMTQSPLSLPVTPGEPASISCRSSQTIVHNNGNTYLEWYLQKPGQSPQLLIYKVSNRF
SGVPDRFSGSGSGTDFTLKISRVEAEDVGVYYCFQGSHVPFTFGQGTKLEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC (SEQ ID NO: 4)
CDRs
VH-CDR-1 (Chothia): GFSLTSYGVH (SEQ ID NO: 5)
VH-CDR-1 (Kabat): SYGVH (SEQ ID NO: 15)
VH-CDR-2 (Chothia): VIWRGGSTDYSAAF (SEQ ID NO: 6)
VH-CDR-2 (Kabat): VIWRGGSTDYSAAFMS (SEQ ID NO: 16)
VH-CDR-3 (Kabat/Chothia): LLRSVGGFAD (SEQ ID NO: 7)
VL-CDR-1 (Kabat/Chothia): RSSQTIVHNNGNTYLE (SEQ ID NO: 8)
VL-CDR-2 (Kabat/Chothia): KVSNRFS (SEQ ID NO: 9)
VL-CDR-3 (Kabat/Chothia): FQGSHVPFT (SEQ ID NO: 10)

Table 3A lists underlined sequences as CDR sequences according to Chothia nomenclature and bold sequences as CDR sequences according to Kabat nomenclature. CDR1, CDR2, and CDR3 are shown in standard order of appearance from left (N-terminus) to right (C-terminus).

B. Nucleotide Sequences Encoding BAN0805 Heavy and Light Chains
BAN0805 HC gene with signal sequence (SEQ ID NO: 11)
ATGGAATGGTCCTGGGTGTTCCTGTTCTTCCTGTCCGTGACCACCGGCGTGCACTCT
CAGGTTCAGCTGCAAGAGTCTGGCCCTGGCCTGGTCAAGCCTTCCGAAACACTGTCT
CTGACCTGCACCGTGTCCGGCTTCTCCCTGACATCTTATGGGGTGCACTGGATCAGA
CAGCCTCCAGGCAAAGGCCTGGAATGGATCGGAGTGATTTGGAGAGGCGGCTCCAC
CGATTACTCCGCCGCCTTCATGTCCCGGCTGACCATCTCTAAGGACACCTCCAAGAA
CCAGGTGTCCCTGAAGCTGTCCTCTGTGACCGCTGCTGATACCGCCGTGTACTACTG
TGCCAAGCTGCTGAGATCTGTCGGCGGCTTTGCTGATTGGGGCCAGGGCACAATGGT
CACCGTGTCTAGCGCTTCTACAAAGGGCCCAAGCGTGTTCCCCCTGGCCCCCTGCTC
CAGAAGCACCAGCGAGAGCACAGCCGCCCTGGGCTGCCTGGTGAAGGACTACTTCC
CCGAGCCCGTGACCGTGTCCTGGAACAGCGGAGCCCTGACCAGCGGCGTGCACACC
TTCCCCGCCGTGCTGCAGAGCAGCGGCCTGTACAGCCTGAGCAGCGTGGTGACCGT
GCCCAGCAGCAGCCTGGGCACCAAGACCTACACCTGTAACGTGGACCACAAGCCCA
GCAACACCAAGGTGGACAAGAGGGTGGAGAGCAAGTACGGCCCACCCTGCCCCCCC
TGCCCAGCCCCCGAGTTCCTGGGCGGACCCAGCGTGTTCCTGTTCCCCCCCAAGCCC
AAGGACACCCTGATGATCAGCAGAACCCCCGAGGTGACCTGTGTGGTGGTGGACGT
GTCCCAGGAGGACCCCGAGGTCCAGTTCAACTGGTACGTGGACGGCGTGGAGGTGC
ACAACGCCAAGACCAAGCCCAGAGAGGAGCAGTTTAACAGCACCTACCGGGTGGTG
TCCGTGCTGACCGTGCTGCACCAGGACTGGCTGAACGGCAAAGAGTACAAGTGTAA
GGTCTCCAACAAGGGCCTGCCAAGCAGCATCGAAAAGACCATCAGCAAGGCCAAGG
GCCAGCCTAGAGAGCCCCAGGTCTACACCCTGCCACCCAGCCAAGAGGAGATGACC
AAGAACCAGGTGTCCCTGACCTGTCTGGTGAAGGGCTTCTACCCAAGCGACATCGCC
GTGGAGTGGGAGAGCAACGGCCAGCCCGAGAACAACTACAAGACCACCCCCCCAGT
GCTGGACAGCGACGGCAGCTTCTTCCTGTACAGCAGGCTGACCGTGGACAAGTCCA
GATGGCAGGAGGGCAACGTCTTTAGCTGCTCCGTGATGCACGAGGCCCTGCACAAC
CACTACACCCAGAAGAGCCTGAGCCTGTCCCTGGGCTGATGA
BAN0805 LC gene with signal sequence (SEQ ID NO: 12)
ATGTCTGTGCCTACACAGGTTCTGGGACTGCTGCTGCTGTGGCTGACCGACGCCAGA
TGCGACATCGTGATGACCCAGTCTCCACTGAGCCTGCCTGTGACACCTGGCGAGCCT
GCTTCCATCTCCTGCAGATCCTCTCAGACCATCGTGCACAACAACGGCAACACCTAC
CTGGAATGGTATCTGCAGAAGCCCGGCCAGTCTCCTCAGCTGCTGATCTACAAGGTG
TCCAACCGGTTCTCTGGCGTGCCCGACAGATTTTCCGGCTCTGGCTCTGGCACCGAC
TTCACCCTGAAGATCTCCAGAGTGGAAGCCGAGGACGTGGGCGTGTACTACTGCTTC
CAAGGCTCTCACGTGCCCTTCACCTTTGGCCAGGGCACCAAGCTGGAAATCAAGCGT
ACGGTGGCCGCTCCCAGCGTGTTCATCTTCCCCCCAAGCGACGAGCAGCTGAAGAG
CGGCACCGCCAGCGTGGTGTGTCTGCTGAACAACTTCTACCCCAGGGAGGCCAAGG
TGCAGTGGAAGGTGGACAACGCCCTGCAGAGCGGCAACAGCCAGGAGAGCGTCAC
CGAGCAGGACAGCAAGGACTCCACCTACAGCCTGAGCAGCACCCTGACCCTGAGCA
AGGCCGACTACGAGAAGCACAAGGTGTACGCCTGTGAGGTGACCCACCAGGGCCTG
TCCAGCCCCGTGACCAAGAGCTTCAACAGGGGCGAGTGCTGATGA
BAN0805 VH gene with signal sequence (SEQ ID NO: 13)
ATGGAATGGTCCTGGGTGTTCCTGTTCTTCCTGTCCGTGACCACCGGCGTGCACTCT
CAGGTTCAGCTGCAAGAGTCTGGCCCTGGCCTGGTCAAGCCTTCCGAAACACTGTCT
CTGACCTGCACCGTGTCCGGCTTCTCCCTGACATCTTATGGGGTGCACTGGATCAGA
CAGCCTCCAGGCAAAGGCCTGGAATGGATCGGAGTGATTTGGAGAGGCGGCTCCAC
CGATTACTCCGCCGCCTTCATGTCCCGGCTGACCATCTCTAAGGACACCTCCAAGAA
CCAGGTGTCCCTGAAGCTGTCCTCTGTGACCGCTGCTGATACCGCCGTGTACTACTG
TGCCAAGCTGCTGAGATCTGTCGGCGGCTTTGCTGATTGGGGCCAGGGCACAATGGT
CACCGTGTCTAGCGC
BAN0805 VL gene with signal sequence (SEQ ID NO: 14)
ATGTCTGTGCCTACACAGGTTCTGGGACTGCTGCTGCTGTGGCTGACCGACGCCAGA
TGCGACATCGTGATGACCCAGTCTCCACTGAGCCTGCCTGTGACACCTGGCGAGCCT
GCTTCCATCTCCTGCAGATCCTCTCAGACCATCGTGCACAACAACGGCAACACCTAC
CTGGAATGGTATCTGCAGAAGCCCGGCCAGTCTCCTCAGCTGCTGATCTACAAGGTG
TCCAACCGGTTCTCTGGCGTGCCCGACAGATTTTCCGGCTCTGGCTCTGGCACCGAC
TTCACCCTGAAGATCTCCAGAGTGGAAGCCGAGGACGTGGGCGTGTACTACTGCTTC
CAAGGCTCTCACGTGCCCTTCACCTTTGGCCAGGGCACCAAGCTGGAAATCAAG
BAN0805 HC gene (SEQ ID NO: 17)
CAGGTTCAGCTGCAAGAGTCTGGCCCTGGCCTGGTCAAGCCTTCCGAAACACTGTCT
CTGACCTGCACCGTGTCCGGCTTCTCCCTGACATCTTATGGGGTGCACTGGATCAGA
CAGCCTCCAGGCAAAGGCCTGGAATGGATCGGAGTGATTTGGAGAGGCGGCTCCAC
CGATTACTCCGCCGCCTTCATGTCCCGGCTGACCATCTCTAAGGACACCTCCAAGAA
CCAGGTGTCCCTGAAGCTGTCCTCTGTGACCGCTGCTGATACCGCCGTGTACTACTG
TGCCAAGCTGCTGAGATCTGTCGGCGGCTTTGCTGATTGGGGCCAGGGCACAATGGT
CACCGTGTCTAGCGCTTCTACAAAGGGCCCAAGCGTGTTCCCCCTGGCCCCCTGCTC
CAGAAGCACCAGCGAGAGCACAGCCGCCCTGGGCTGCCTGGTGAAGGACTACTTCC
CCGAGCCCGTGACCGTGTCCTGGAACAGCGGAGCCCTGACCAGCGGCGTGCACACC
TTCCCCGCCGTGCTGCAGAGCAGCGGCCTGTACAGCCTGAGCAGCGTGGTGACCGT
GCCCAGCAGCAGCCTGGGCACCAAGACCTACACCTGTAACGTGGACCACAAGCCCA
GCAACACCAAGGTGGACAAGAGGGTGGAGAGCAAGTACGGCCCACCCTGCCCCCCC
TGCCCAGCCCCCGAGTTCCTGGGCGGACCCAGCGTGTTCCTGTTCCCCCCCAAGCCC
AAGGACACCCTGATGATCAGCAGAACCCCCGAGGTGACCTGTGTGGTGGTGGACGT
GTCCCAGGAGGACCCCGAGGTCCAGTTCAACTGGTACGTGGACGGCGTGGAGGTGC
ACAACGCCAAGACCAAGCCCAGAGAGGAGCAGTTTAACAGCACCTACCGGGTGGTG
TCCGTGCTGACCGTGCTGCACCAGGACTGGCTGAACGGCAAAGAGTACAAGTGTAA
GGTCTCCAACAAGGGCCTGCCAAGCAGCATCGAAAAGACCATCAGCAAGGCCAAGG
GCCAGCCTAGAGAGCCCCAGGTCTACACCCTGCCACCCAGCCAAGAGGAGATGACC
AAGAACCAGGTGTCCCTGACCTGTCTGGTGAAGGGCTTCTACCCAAGCGACATCGCC
GTGGAGTGGGAGAGCAACGGCCAGCCCGAGAACAACTACAAGACCACCCCCCCAGT
GCTGGACAGCGACGGCAGCTTCTTCCTGTACAGCAGGCTGACCGTGGACAAGTCCA
GATGGCAGGAGGGCAACGTCTTTAGCTGCTCCGTGATGCACGAGGCCCTGCACAAC
CACTACACCCAGAAGAGCCTGAGCCTGTCCCTGGGCTGA
BAN0805 LC gene (SEQ ID NO: 18)
GACATCGTGATGACCCAGTCTCCACTGAGCCTGCCTGTGACACCTGGCGAGCCTGCT
TCCATCTCCTGCAGATCCTCTCAGACCATCGTGCACAACAACGGCAACACCTACCTG
GAATGGTATCTGCAGAAGCCCGGCCAGTCTCCTCAGCTGCTGATCTACAAGGTGTCC
AACCGGTTCTCTGGCGTGCCCGACAGATTTTCCGGCTCTGGCTCTGGCACCGACTTC
ACCCTGAAGATCTCCAGAGTGGAAGCCGAGGACGTGGGCGTGTACTACTGCTTCCA
AGGCTCTCACGTGCCCTTCACCTTTGGCCAGGGCACCAAGCTGGAAATCAAGCGTAC
GGTGGCCGCTCCCAGCGTGTTCATCTTCCCCCCAAGCGACGAGCAGCTGAAGAGCG
GCACCGCCAGCGTGGTGTGTCTGCTGAACAACTTCTACCCCAGGGAGGCCAAGGTG
CAGTGGAAGGTGGACAACGCCCTGCAGAGCGGCAACAGCCAGGAGAGCGTCACCG
AGCAGGACAGCAAGGACTCCACCTACAGCCTGAGCAGCACCCTGACCCTGAGCAAG
GCCGACTACGAGAAGCACAAGGTGTACGCCTGTGAGGTGACCCACCAGGGCCTGTC
CAGCCCCGTGACCAAGAGCTTCAACAGGGGCGAGTGCTGA
BAN0805 VH gene (SEQ ID NO: 19)
CAGGTTCAGCTGCAAGAGTCTGGCCCTGGCCTGGTCAAGCCTTCCGAAACACTGTCT
CTGACCTGCACCGTGTCCGGCTTCTCCCTGACATCTTATGGGGTGCACTGGATCAGA
CAGCCTCCAGGCAAAGGCCTGGAATGGATCGGAGTGATTTGGAGAGGCGGCTCCAC
CGATTACTCCGCCGCCTTCATGTCCCGGCTGACCATCTCTAAGGACACCTCCAAGAA
CCAGGTGTCCCTGAAGCTGTCCTCTGTGACCGCTGCTGATACCGCCGTGTACTACTG
TGCCAAGCTGCTGAGATCTGTCGGCGGCTTTGCTGATTGGGGCCAGGGCACAATGGT
CACCGTGTCTAGCGC
BAN0805 VL gene (SEQ ID NO: 20)
GACATCGTGATGACCCAGTCTCCACTGAGCCTGCCTGTGACACCTGGCGAGCCTGCT
TCCATCTCCTGCAGATCCTCTCAGACCATCGTGCACAACAACGGCAACACCTACCTG
GAATGGTATCTGCAGAAGCCCGGCCAGTCTCCTCAGCTGCTGATCTACAAGGTGTCC
AACCGGTTCTCTGGCGTGCCCGACAGATTTTCCGGCTCTGGCTCTGGCACCGACTTC
ACCCTGAAGATCTCCAGAGTGGAAGCCGAGGACGTGGGCGTGTACTACTGCTTCCA
AGGCTCTCACGTGCCCTTCACCTTTGGCCAGGGCACCAAGCTGGAAATCAAG

The sequences encoding a signal peptide are underlined. The start codons are in bold and the stop codons are in italic.

What is claimed is:

1.-25. (canceled)

26. A method of producing an assembled antibody, comprising the steps of:
(1) providing one or more vectors, wherein the one or more vectors comprise a first nucleic acid sequence comprising SEQ ID NO:19 and a second nucleic acid sequence comprising SEQ ID NO: 20;
(2) transfecting a host cell with the one or more vectors; and
(3) culturing the host cell under conditions which allow for the secretion of the assembled antibody.

27. The method of claim 26, wherein the first nucleic acid sequence comprising SEQ ID NO: 19 comprises SEQ ID NO:17 and the second nucleic acid sequence comprising SEQ ID NO:20 comprises SEQ ID NO:18.

28. The method of claim 26, wherein the first nucleic acid sequence comprises a signal peptide and the second nucleic acid sequence comprises a signal peptide.

29. The method of claim 28, wherein the first nucleic acid sequence comprising SEQ ID NO: 19 comprises SEQ ID NO:13 and the second nucleic acid sequence comprising SEQ ID NO:20 comprises SEQ ID NO:14.

30. The method of claim 29, wherein the first nucleic acid sequence comprising SEQ ID NO: 19 comprises SEQ ID NO:11 and the second nucleic acid sequence comprising SEQ ID NO:20 comprises SEQ ID NO:12.

31. The method of claim 26, wherein the one or more vectors is one vector.

32. The method of claim 31, wherein the one vector is a double gene vector.

33. The method of claim 26, wherein the transfection is transient transfection.

34. The method of claim 26, wherein the host cell is CHOK1SV GS-KO cell.

35. The method of claim 26, further comprising the step of purification by affinity chromatography.

36. The method of claim 35, wherein the affinity chromatography is Protein A affinity chromatography.

37. The method of claim 26, wherein the antibody comprises a heavy chain comprising the amino acid sequence of SEQ ID NO:1 and a light chain comprising the amino acid sequence of SEQ ID NO:2.

38. The method of claim 37, wherein the antibody comprises a heavy chain comprising the amino acid sequence of SEQ ID NO:3 and a light chain comprising the amino acid sequence of SEQ ID NO:4.

39. The method of claim 37 or 38, wherein the antibody comprises two heavy chains and two light chains.
